(19) United States
(12) Patent Application Publication
(10) Pub. No.: US 2025/0369030 A1
(43) Pub. Date: Dec. 4, 2025

(54) METHOD FOR SAMPLING MICROORGANISMS ON INDUSTRIAL AGRI-FOOD SURFACES

(71) Applicant: REALCO, Louvain-la-neuve (BE)

(72) Inventors: Laurent DELHALLE, Esneux (BE); Sébastien FASTREZ, Nouvelles (BE)

(73) Assignee: REALCO, Louvain-la-neuve (BE)

(21) Appl. No.: 18/874,063

(22) PCT Filed: Jun. 20, 2023
(86) PCT No.: PCT/EP2023/066576

§ 371 Date: Dec. 11, 2024

(30) Foreign Application Priority Data
Jun. 20, 2022 (BE) BE2022/5483

(51) Int. Cl. C12Q 1/04; C12Q 1/24

(52) U.S. Cl. CPC C12Q 1/04; C12Q 1/24

(57) ABSTRACT

A method is for collecting microorganisms from surfaces, for subsequent laboratory detection and analysis.

### TECHNICAL FIELD

This invention relates to a method for sampling, removing, collecting and preserving microorganisms on surfaces (industrial and/or agri-food and/or communities), which is non-lethal and compatible with several subsequent tests, for subsequent detection and laboratory analysis.

### PRIOR ART

In many fields of activity such as the agri-food sector, communities, medical and veterinary sectors, problematic contamination due to the presence of microorganisms is very frequently observed.

Traditionally, in hospitals and in medical or veterinary practices, the presence of microorganisms is responsible for nosocomial diseases while in the agri-food sector and in communities, these microorganisms are responsible for the degradation of perishable goods but also for the transfer of contaminants to consumers of products from, for example, a meat, fruit, vegetable or other production chain.

However, these days, strict hygiene standards are required and it is therefore necessary to ensure that the number of microorganisms responsible for such contamination is kept below an acceptable threshold value and this acceptable threshold value is specific to each field of activity.

Planktonic bacteria are a first example of contaminating microorganisms, these bacteria are free at the level of a liquid or solid substrate and pose a problem per se because they are able to directly contaminate any type of surface such as foodstuffs, medical tools, conveyor belts, storage tanks or even human patients or animals themselves.

However, today, it is widely recognised that the problems related to contamination by microorganisms are all the more pressing as they form biofilms.

Indeed, in any type of industry and more particularly in the field of the agri-food industry, biofilms are inevitably formed, given the richness of the surrounding environment.

Biofilms are defined as a consortium of microorganisms embedded in a matrix of extracellular polymeric substances. In other words, biofilms are viscous films that develop on all surfaces, following the adhesion of microorganisms to these surfaces and the secretion by these of polymers covering them, facilitating their adhesion to the surface and therefore forming the extracellular matrix. Biofilms therefore constitute a protective layer around microorganisms that is very resistant to chemical and thermal attack, which makes eliminating them using conventional biocides very difficult and they are also a recurring and significant source of contamination of the surrounding environment.

This resistance is explained on the one hand by the matrix of extracellular polymeric substances that forms a physical barrier to the diffusion of biocide molecules in the biofilm and, on the other hand, by the sessile state of microorganisms and their ability to very rapidly exchange genes responsible for certain biocide resistance mechanisms.

It is therefore easy to understand that biofilms are very resistant and extremely varied structures, for example different strains of the same bacteria can form different biofilms, and these biofilms are even more varied as they are composed of several different bacteria. In addition, bacteria exchange resistance genes between them and the environment also influences the biofilm, further increasing their complexity and resistance.

In addition, the extracellular matrix of biofilms can be identified when it is highly developed but in the majority of cases, the biofilm develops insidiously in the facilities and its presence will be detected only when the quality of the finished product is analysed.

With regard to the growth of the biofilm, this takes place in a cyclical manner, comprising a growth phase during which the accumulation of microorganisms occurs and a detachment phase during which whole pieces of biofilms and microorganisms detach themselves by erosion and under the effect of their weight to contaminate surfaces, foodstuffs, medical tools, conveyor belts, storage tanks or even human patients, consumers or animals themselves.

This leads to the manufacturer having to stop their production line and carry out a cleaning cycle to eliminate the biofilm, involving many hours of work and resources deployed and a loss of yield.

From the foregoing, it is shown that contaminating microorganisms and/or biofilms are a real issue, particularly in the field of healthcare (hospitals, dental or medical practices), veterinary care and the agri-food industry. This problem is all the more critical since microorganisms and/or biofilms can involve bacteria responsible for potentially fatal infections in individuals, whether these bacteria are present in hospitals, veterinary practices or in the agri-food industry and are ultimately found on food products intended for consumption.

As indicated above, the issue of microorganisms and more particularly biofilms is twofold. On the one hand, conventional disinfectants and biocides are very often ineffective because they fail to reach the microorganisms protected by the extracellular matrix of the biofilm, with this matrix having a complex and highly variable structure and composition. On the other hand, a biofilm is generally mixed, i.e. it contains a multitude of different bacteria or the same bacteria but which are of different strains, which promotes the spread of resistance genes between the bacteria of the biofilm and therefore makes the treatment thereof very difficult or even ineffective.

Consequently, from one environment to another or from one sector of activity to another, it is rather common for the biofilms detected to differ completely. In order to detect biofilms, there are kits for detecting the presence of biofilms on surfaces or in more specific facilities (water circuits, etc.); these kits (such as the one disclosed in document EP2537601) allow the performance of the selective staining of biofilms. It is therefore currently possible to determine areas where biofilms are present in order to eliminate them without knowing the precise nature of the microorganisms (bacteria) causing the target biofilm.

As a result, biofilm removal compositions comprising several enzymes are used without knowing whether the enzymes used and optionally formulated in a detergent base (see for example document EP2243821) are actually suitable to act on a given biofilm. For this reason, current treatments are rather random and non-specific, which is economically unprofitable and can lead to wasted time and a lengthy downtime of facilities.

Furthermore, techniques for collecting microorganisms from surfaces have been developed in order to characterise and/or quantify the populations of microorganisms present on surfaces and responsible for contamination. These collection methods therefore make it possible to determine, on a surface, the different types (strains) of bacteria and microorganisms present.

Among the methods for collecting microorganisms found on a surface, one of the references in this area is the ISO 18593:2004(F) Standard, which provides and defines horizontal methods for collection techniques on surfaces (more specifically on surfaces found in the context of the agri-food industry). The “cloth/sponge” method is an example of collection techniques cited in this Standard, with this method making it possible to search for and/or count microorganisms present on a surface. Briefly, this collection method involves wetting the cloth/sponge with a quantity of diluent which is sterile physiological serum, sampling the surface in two perpendicular directions before introducing the cloth/sponge into a sterile container with the diluent. Subsequently, after optional storage of the sample, the latter is analysed quantitatively and/or qualitatively.

Unfortunately, though such a method ensures the collection of free microorganisms on the surface of a substrate, i.e. the collection of planktonic microorganisms, it turns out to be ineffective when the surface is contaminated by microorganisms that have formed a biofilm. Indeed, as explained above, the microorganisms protected by the extracellular matrix of the biofilm, with this matrix having a complex and highly variable structure and composition and with the biofilms being mixed, are even more varied and complex as they are composed of several different bacteria or different strains.

In addition, it turns out that collection techniques based on a water-impregnated medium can have a beneficial effect on the development of microorganisms, even when performing a collection from a surface. Indeed, it is recognised that certain bacteria exhibit increased growth in the presence of water, the water-impregnated medium therefore promoting this growth during collection but also after said collection since a thin film of water remains on the treated media where collection took place. This can therefore promote the development of certain microorganisms that are not collected or because they are protected by a biofilm.

In order to overcome the disadvantages of water-impregnated collection media, document DE10304331 is known from the prior art, which discloses an enzymatic preparation that can be used to eliminate biofilms from surfaces in the absence of biocides. This enzymatic preparation comprises one or more enzymes from the group of polysaccharidases and/or proteases and optionally nucleases.

Unfortunately, even though the compositions according to document DE10304331 have a certain effectiveness in terms of the collection of microorganisms, it appears that they are insufficient given the diversity and complexity of microorganisms and biofilms that may be present on a surface of a facility and that they do not allow for a representative collection of the bacterial populations that are nevertheless present there, i.e. they only allow for a low coverage of microorganisms.

Indeed, the biofilms and/or microorganisms present on a surface of a facility are on the one hand composed of a varied and complex population of microorganisms, i.e. different microorganisms but these different microorganisms may also still be of different strains, thus leading to different resistances but also the formation of varied, complex and different extracellular matrices of biofilms, sometimes within the same surface of a facility.

### BRIEF SUMMARY OF THE INVENTION

A first object of the present invention relates to a method for sampling, removing, collecting and preserving microorganisms on surfaces, for subsequent detection and laboratory analysis comprising the following steps:
performing the sterile and calibrated application of a composition for collecting microorganisms comprising one or more surfactants, and at least two enzymes chosen from the group comprising a protease, a polysaccharidase, a laccase, a lipase, a cellulase and a mannanase, and a sequestering agent to a surface to be sampled,
leaving said composition to act on said surface to be sampled for a predetermined period of time so as to recover a sample of microorganisms,
performing the sterile collection of said composition containing said sample of microorganisms,
depositing said composition containing said sample of microorganisms in at least one sterile container, preferably comprising a solution for preserving said collection solution, said sterile container containing said sample of microorganisms, for the preservation of said sample for subsequent detection and laboratory analysis,
said method is characterised in that said surfactant(s) present in said collection composition comprise(s) an anionic surfactant, or consist (essentially) of one or more anionic surfactants, and in that the overall content of said (anionic) surfactants is between 0.01% and 10% by weight (weight of the surfactant(s): weight of the collection composition).

Advantageously, said calibrated application of said collection composition is a calibrated application of a controlled volume of said collection composition, preferably a calibrated spraying of a controlled volume of said collection composition.

Preferably, said calibrated application of a controlled volume of said collection composition is an application of a content of between 0.5 and 5 millilitres of composition per application on a surface of 100 cm², preferably of a content of between 0.7 and 3 millilitres of composition per application on a surface of 100 cm², preferentially between 0.9 and 2 millilitres of composition per application on a surface of 100 cm², advantageously between 1 and 1.5 millilitres of composition per application on a surface of 100 cm².

Preferably, in this method, the step of leaving said collection composition to act takes place for a period of at least 10 seconds, at least 20 seconds, at least 30 seconds, at least 1 minute, preferably at least 2 minutes, preferentially at least 3 minutes, advantageously at least 4 minutes, particularly advantageously at least 5 minutes and at most 15 minutes, preferably at most 10 minutes, preferably the time is approximately 5 minutes.

Advantageously, the step of sterile collection of said composition containing said sample of microorganisms comprises a first step of contacting at least one means of sterile harvesting of said microorganisms on said surface to be sampled.

Preferably, the sterile harvesting means is chosen from the group consisting of a tissue, a wipe, a swap [sic], a sponge, a scraper and a swab.

Advantageously, a step of storing said sterile container containing said sample of microorganisms is carried out at a temperature below 15° C., preferably below 10° C., advantageously below 5° C. between 4 and 8° C.

Preferably, the surfactants (present in the collection composition used in the method of the invention) are present at a content 25 of at least 0.15% by weight relative to the weight of said composition, preferably at a content of at least 0.02% by weight and, preferably at a content of at most 5% by weight, preferably at most 1% by weight, preferably at most 0.5% by weight, or even at most 0.1%.

Preferably, the collection composition (used in the method of the invention) comprises at least three enzymes, preferably at least four enzymes, preferentially at least five enzymes and advantageously at least six enzymes.

Preferably, the collection composition (used in the method of the invention) further comprises at least one stabilising agent present at a content of at least 15% relative to the weight of said composition, said stabiliser being chosen from the group of polyols (glycerol) and propylene glycol.

Preferably, the collection composition (used in the method of the invention) further comprises at least one sequestering agent at a content of at least 0.1% by weight relative to the weight of said composition, preferably at a content of at least 0.2%, preferentially at a content of between 0.3% and 3% by weight, advantageously between 0.3% and 1% by weight, and particularly advantageously between 0.3% and 0.8% by weight, said sequestering agent preferably being a carboxymethyl inulin, a phosphonate, sodium glycolate and mixtures thereof.

Preferably, the collection composition (used in the method of the invention) has previously been sterilised by a physical treatment chosen from electron beam irradiation, gamma ray irradiation and sterilising filtration.

In this method, optionally, the sampling composition comprises a (serine) protease and the preservative solution comprises at least one (serine) protease inhibitor, preferably PMSF.

Preferably, the preservative solution (used in the method of the invention) comprises at least one polyoxyethylene sorbitan derivative and/or at least one phosphoglyceride derivative.

Advantageously, this polyoxyethylene sorbitan derivative is present at a content of at least 4 g/L, preferably at a content of at least 4.3 g/L, preferentially at a content of at least 4.6 g/L, advantageously at a content of at least 4.9 g/L and/or said at least one phosphoglyceride derivative is present at a content of between 0.5 and 3 g/L, preferably at a content of between 0.5 and 2.5 g/L, preferentially between 0.5 and 2 g/L, advantageously between 0.5 and 1.5 g/L.

Preferably, the preservative solution (used in the method of the invention) further comprises at least one nutrient extract, preferably comprising a mixture of peptides.

Advantageously, this nutrient extract (peptide mixture) is present at a content of at least 1 g/L, preferably at least 2 g/L, preferentially at least 3 g/L, advantageously at least 4 g/L and particularly advantageously at least 5 g/L.

Advantageously, this method allows, and therefore further comprises, the step of culturing the microorganisms (e.g. Listeria and/or Salmonella) collected and/or preserved.

Advantageously, this method allows, and therefore (further) comprises the step of specific detection of the microorganisms collected, preserved and/or cultured.

Specific detection is preferably carried out by immunology.

Alternatively, or in addition, specific detection is carried out by genetic analysis.

Alternatively or additionally, the method according to the invention allows and therefore comprises a biochemical test, said biochemical test being an enzymatic test, or involving proteins or enzymes, preferably ATP-metry, NADH measurement, catalase activity measurement and protein residue measurement.

### DETAILED DESCRIPTION OF THE INVENTION

There is therefore an identified need to provide a method that aims to address the disadvantages of the prior art by allowing efficient, representative and adequate collection of microorganism populations, whether they are free or in the form of a biofilm with a complex structure that can vary considerably from one sample to another. In addition, there is a need to provide a composition allowing the collection of microorganisms on different types of surfaces, in different environments, which is stable over time and which is compatible with users or even with the production chain, for example food in an agri-food industry.

To solve this problem, the invention provides for the use of a composition for collecting (and removing) microorganisms comprising:
at least one surfactant representing at least 0.01% by weight relative to the weight of said composition and at most 10% by weight (sum of the weights of the surfactants: total weight of the composition), this surfactant comprising, or even consisting (essentially) of an anionic surfactant,
at least one stabilising agent (glycerol) representing at least 15%, preferably at least 18%, preferentially at least 20% by weight relative to the weight of said composition, and/or preferably less than 30%, or even less than 25%, by weight relative to the weight of said composition,
at least one sequestering agent,
at least two enzymes chosen from the group consisting of a protease, a polysaccharidase, a laccase, a lipase, a cellulase and a mannanase.

The characteristics of the collection composition according to this invention have the advantage of allowing the collection of microorganisms, whether they are in planktonic form or in the form of complex and varied biofilms.

The collection composition, comprising at least two enzymes chosen from the group comprising a protease, a polysaccharidase, a laccase, a lipase, a cellulase and a mannanase, advantageously makes it possible to effectively degrade and deconstruct the extracellular matrix of biofilms and in particular a varied and complex population of biofilms having different extracellular matrices, therefore making it possible to release the bacteria so they can be collected.

Advantageously, the sequestering agent (di- or multivalent cations), is present at a content by weight of at least 0.2%, preferably at least 0.3%, or even at least 0.4%.

The sequestering agent is preferably glucono-delta-lactone, sodium gluconate, potassium gluconate, calcium gluconate, sodium glycolate, citric acid (a citrate), phosphoric acid (a phosphate), tartaric acid (a tartrate), sodium acetate, sorbitol, carboxymethyl inulin (sodium salt), a phosphonate, and mixtures thereof. So-called “mild” sequestering agents are preferred: the inventors have noted that these “mild” sequestering agents allow for better enzymatic activity at the collection level and/or for downstream microbiological tests involving enzymes (e.g., the inventors have noted interference with downstream PCR tests when too much sequestering agent and/or overly strong sequestering agents are present).

In addition, the sequestering agent and the pH of between 5 and 11 of the collection composition advantageously make it possible to form complexes with mineral ions which will be fixed in a form that prevents their precipitation by the usual reactions and therefore to stabilise the composition over time, in particular in view of the enzymes present in such proportions.

In the context of this invention, it has been determined that the collection composition comprising at least one anionic surfactant which represents at least 0.01% by weight relative to the weight of said composition and, preferably less than 10%, or even less than 5%, or even less than 1.5%, or even less than 1% or even less than 0.1%, makes it possible in a particularly advantageous manner to remove and collect a representative and complete proportion of a population of microorganisms present on a surface, whether they are planktonic or in the form of biofilms since the enzymes of the composition according to the invention allow the release thereof. Surprisingly, low surfactant contents are involved.

Preferably, the surfactants are chosen so that, overall, they have an HLB value of between 10 and 16.

Advantageously, the surfactants comprise foaming surfactants, which has the advantage on the one hand of removing and collecting the microorganisms and on the other hand of trapping the microorganisms collected in the composition according to the invention in order to carry out an effective and representative collection of the population of contaminants from a surface.

The collection composition is advantageously capable of adhering correctly to different types of surfaces and in different environments and therefore of allowing an effective, representative and adequate collection of populations of microorganisms, whether they are free but also in the form of a biofilm with a complex and variable structure.

As a result, the collection composition, unlike current compositions, makes it possible to obtain a truly representative and complete collection of the contaminants present on a surface, for different types of surfaces and for different environments. This is particularly advantageous because by having a representative and complete sample of microorganisms, they can subsequently be identified and a targeted and effective elimination treatment, using the appropriate compounds and no longer a cocktail of compounds which is often ineffective, can be applied to the surface. This is of course a considerable advantage in terms of economics and time.

The benefit of the collection composition is therefore twofold in that it makes it possible, on the one hand, to collect and sample all the contaminants from a surface whether they are planktonic or in the form of biofilms for the purpose of analysing them, and that it then makes it possible to determine precisely which treatment will be applied to eliminate these contaminants.

Preferably, the at least one anionic surfactant of the collection composition is sodium laureth sulphate (CAS 9004-82-4).

Preferably, the at least one stabilising agent and/or viscosity agent is chosen from the group comprising a polyol or propane 1,2-diol or propane 1,3-diol. Preferably, the stabilising agent is glycerol.

Preferably, the collection composition has a pH of between 6 and 10, preferably between 7 and 9, preferentially between 8 and 9, particularly advantageously between 8.2 and 8.6.

Preferably, the collection composition is in liquid form, preferably in sprayable liquid form, which allows easy and reproducible application.

Preferably, the collection composition further comprises at least one additional enzyme chosen from the group consisting of oxidoreductases, lyases, transferases, peptidases, lipases, esterases, and glucosaminidases.

Other embodiments of the collection composition according to the invention are indicated in the appended claims.

The invention also relates to the use of a kit for removing, collecting and preserving microorganisms in a living or revivable state for subsequent laboratory analyses.

As explained above, the benefit of the sampling composition is twofold in that it makes it possible, on the one hand, to collect and sample all the contaminants from a surface whether they are planktonic or in the form of biofilms and that it then makes it possible to determine precisely which treatment to eliminate these contaminants will be applied after analyses have been carried out to identify and precisely characterise the contaminants.

Consequently, it is important that the collection composition, which contains the contaminants, microorganisms and biofilms, following collection can allow for a reliable, correct and representative analysis of the population collected without said composition after collection interfering with subsequent analyses.

To solve this problem, this invention provides for the use of a kit for removing, collecting and preserving microorganisms in a living or revivable state for subsequent laboratory analyses comprising:
a composition for removing and collecting microorganisms as described above,
at least one means for the sterile harvesting of said microorganisms,
at least one sterile container, for example a sterile bag, comprising a preservative solution for collecting and preserving microorganisms in a living or revivable state for subsequent laboratory analyses.

Advantageously, the at least one means for the sterile harvesting of the microorganisms of the kit is a tissue, a wipe, a sponge.

The invention therefore relates to a method for removing, collecting and preserving microorganisms in a living or revivable state for subsequent laboratory analyses comprising the following steps:
a) at least one controlled (calibrated) (and sterile) application of a composition for collecting (and removing) microorganisms as described above,
b) leaving said collection composition to act on said surface to be sampled,
c) depositing at least one means for harvesting said microorganisms on said surface to be sampled,
d) leaving said at least one means for harvesting to be soaked by said removal composition,
e) performing a sterile collection of said at least one means for collecting the soaked microorganisms and depositing it in at least one sterile container, for example a sterile bag, preferably comprising a preservative solution for collecting and preserving microorganisms in a living or revivable state for subsequent laboratory analyses.

Advantageously, step b) takes place for a period of at least 10 seconds, at least 20 seconds, at least 30 seconds, at least 1 minute, preferably at least 2 minutes, preferentially at least 3 minutes, advantageously at least 4 minutes, particularly advantageously at least 5 minutes.

Optionally, the method according to the invention further comprises at least one preliminary step of preheating said removal composition, preferably at a temperature of between 20 and 60° C., preferably between 30 and 50° C., advantageously between 40 and 50° C.

Advantageously, the method according to the invention further comprises at least one step of storing said sterile container containing said soaked harvesting means, at a temperature of less than 15° C., preferably less than 10° C., advantageously less than 5° C.

Advantageously, the preservative composition comprises one or 2 or all 3 compounds selected from the group of protease inhibitors, a nutrient (e.g. protein hydrolysates and/or a mixture of peptides) and preservative inhibitors selected from a polyoxyethylene sorbitan derivative (e.g. polyoxyethylene sorbitan monooleate) and/or at least one phosphoglyceride derivative (e.g. phosphatidyl choline).

Preferably, the preservative inhibitors are present at a content of at least 4 g/litre, preferably at a content of at least 4.3 g/L, preferentially at a content of at least 4.6 g/L, advantageously at a content of at least 4.9 g/L.

The protease inhibitors are preferably absent. When present, they are preferably chosen from the group consisting of sulfonylation agents (e.g. AEBSF, PMSF, DFP), chelating agents (e.g. EDTA), alkylating agents (e.g. fluoromethyl, chloromethyl and acyloxymethyl ketones, epoxides, aziridines, vinyl sulfones, and other Michael acceptors), acylating agents (e.g. β-lactams, lactones, aza-peptides, heterocyclic derivatives), phosphonylation agents (e.g. DFP), phosphonyl or sulfonyl fluorides, thiadiazoles, amino acid sulphate salts and tissue inhibitors of matrix metalloproteinases (TIMPs).

When the collection composition comprises a serine protease, PMSF (PhenylMethylFulfonyl Fluoride) is the preferred protease inhibitor, despite the limitations associated with its use.

This method advantageously makes it possible to carry out a step of culturing the harvested (collected) and/or preserved microorganisms, and/or to carry out a biochemical test, involving enzymes and/or proteins.

In addition, or as an alternative, this method makes it possible to carry out a step of specifically labelling the harvested (collected) and/or cultured microorganisms, including Listeria sp. and/or Salmonella sp.

This specific labelling is advantageously carried out by an immunological test and/or by a genetic test.

In the context of the present invention, an immunological test is preferably understood to mean any test comprising one or more antibodies capable of binding specifically to a component of a given bacterium. Such an immunological test is therefore qualitative (identity of the bacterial strain, presence of a given factor) and/or quantitative. This test may comprise secondary labelling means, such as fluorescent labelling means, which allow rapid and precise analysis.

In the context of this invention, a genetic test is preferably understood to mean any test where the presence, or even the abundance, of a target nucleic acid molecule in the sample is analysed. The preferred genetic tests are sequencing and PCR (Polymerase Chain Reaction) or other types of genetic amplification (including isothermal amplification techniques) involving probes and/or primers specific to the microorganism(s) to be identified (e.g. Listeria and/or Salmonella, including specific detection of pathogenic strains of Listeria or Salmonella, or specific detection of problematic strains, relative to other microorganisms present, but not problematic).

### DETAILED DESCRIPTION OF AN EMBODIMENT OF THE INVENTION

Other characteristics and advantages of this invention will be set forth in the non-limiting description below, and with reference to the drawings and the examples.

### EXAMPLES

It is understood that this invention is in no way limited to the embodiments described above and that many modifications can be made thereto without departing from the scope of the appended claims.

### Example 1

A meat processing company located in Belgium was chosen for this study. All the facilities were previously cleaned and disinfected before performing the collections.

The removal protocol is applied with limited mechanical actions that could introduce a reproducibility error; it includes the following steps:

Quickly scan (screen) the areas to undergo collection with the UV lamp and BDK detection kit (Realco);
Preheat the collection solution to 45° C.;
Place two templates on the surfaces to undergo collection (in order to define a surface of 10 cm by 10 cm): physiological water vs. collection composition (14 sprays to cover the area; 1.2 g/spray);
Leave the collection solution to act for 5 minutes;
Place the sterile wipe on the two collection areas and leave it to soak;
Perform sterile collection using forceps and place in a sterile transport bag;
Store the samples in refrigerated containers at 4° C.;
Then evaluate the two areas with the lamp and the BDK.

A total of 6 collection areas were included in this study: Zone 1 round table; Zone 2 lid for rubbish table; Zone 3 stainless steel bin; Zone 4 saw table; Zone 5 plastic surface; Zone 6 derinding machine.

In these 6 zones, the collection composition comprising enzymes (including a subtilisin, activity equivalent to 10 Anson Units/kg) and detergents (approximately 10% by weight) allows a much better recovery of microorganisms, both in classical microbiology and by ATP measurement (2G) or by culturing. Conversely, there were no more microorganisms on the surfaces where the collection solution was placed, unlike the surface treated with physiological water. The difference is especially obvious at the level of classical microbiology, compared with ATP 2G or by testing involving a culturing step (little difference noted).

The inventors reproduced the example, to include metagenomic analyses, while varying the contact time of the collection composition on the surface (30 seconds and 5 minutes). Again, the enzymatic composition allows for much better collection (e.g. 100 times better in classical microbiology and 14 times better in qPCR). The bacteria found show an increased proportion of Pseudomonas.

### Example 2—Addition of a Preservative Solution; Salmonella

The inventors, suspecting harmful effects associated with the collection composition, sought to determine to what extent an adjuvant solution could counteract these, in the context of tests aimed at determining the presence of Salmonella and/or Listeria, either by PCR or qPCR, or by using commercial detection kits, for example those from BioMérieux. To do this, a so-called ‘preservative solution’ was tested (in g/l): Proteins (beef extract; 5); Peptone, 10; Polysorbate 80 (Tween 80; 5); Lecithin 0.7; NaCl, 5. In addition, various serine protease inhibitors were used, in order to neutralise the subtilisin activity of the collection solution.

The first series of tests was performed in the laboratory, where known quantities of bacteria (0.1 ml of 10⁷ CFU/ml Salmonella) were incubated either in physiological solution (100 ml), or in the collection composition of Example 1 (6 ml+94 ml physiological water), or in the composition of Example 1, before the preservative solution was added (6 ml collection composition, 90 ml water, 4 ml preservative solution).

When 10⁷ UFC/ml of Salmonella are analysed (i.e. 10⁴ CFU/ml in the final mixture), the different situations are compatible with the identification of the microorganism by means of PCR analyses or the Vidas® Salmonella test. However, detection by the Vidas SLM® test (culture broth, then specific measurement by antigenic test and fluorescence; this test provides information on the absence or presence of Salmonella) fails when the collection composition is present, whereas it works when the bacteria are diluted in physiological water or when the preservative solution has been added.

Furthermore, in this example, the inventors consider that the Salmonella concentrations are high, which does not necessarily, or even rarely, represent the situation in the field, where even 1 CFU must be detected.

### Example 3—Addition of a Preservative Solution; Listeria

In this case, the enzymatic solution causes problems at the detection level (enumeration and qPCR); these problems are reduced thanks to the preservative solution, however the commercial Vidas® kit (LMX) still does not work.

Furthermore, when the amount of Listeria inoculated is reduced, the microorganism is generally no longer detected following mixing (Vidas LMX®, never; Vidas LMO2®, not every time) with the collection composition, while detection remains strong when mixing was carried out in physiological water only. In this case (which is intentionally extreme), the preservative composition did not improve detection. As a precaution, the tests were reproduced and three strains of Listeria were tested with the same results every time; these strains were ATCC 19114, NCTC 11994 and ATCC 13932.

By comparing all the results, the inventors conclude that the collection composition slows the growth of microorganisms (Listeria), a problem that is partially solved by the preservative composition. On the other hand, the collection composition interferes with certain downstream microbiological tests. Since the nature and abundance of microorganisms on a surface to be tested is unknown in advance, the inventors therefore conclude that the compositions of Examples 1 to 3 are useful, but would benefit from being further improved.

### Example 4—Identifying Harmful Compounds

The inventors investigated various commercial enzymes to see if any compounds interfered with downstream microbiological tests. The inventors noticed that the presence of compounds such as phenoxyethanol, potassium sorbate or thiazolinone derivatives is quite widespread. Thus, the inventors selected enzymatic compositions which are free from such compounds, or insofar as possible, even though the inventors found significant concentrations of 2-phenoxyethanol in many commercial enzymatic compositions. The compounds tested included Bronopol, triclosan, idocarb, chlorhexidine, DCOIT, BIT, OIT, CIT, MIT, ethylparaben, methylparaben, butylparaben, phenylparaben, isopropylparaben, isobutylparaben, propylparaben, salisylic acid, 4-hydroxybenzoic acid, 4-methylbenzoic acid, benzoic acid, sorbic acid, dehydroacetic acid, 1-phenoxypropan-2-ol, 1,2-dibromo-2,4-dicyanobutane, 2-hydroxybiphenyl, 2-phenoxyethanol (found in highest quantities, sometimes up to 0.4% by weight), bensyl alcohol and chlorophenesin.

### Comparative Example 1

Suspecting interference between the proteases in the collection composition and the enzymes or antibodies in the downstream tests, the inventors tested the effect of adding protease inhibitors to the preservative solution (see example 3), while avoiding commercial enzymatic compositions comprising compounds identified as harmful (see example 4) as much as possible.

Different ways of applying the protease inhibitor were tested. Since the chosen protease was a subtilisin or trypsin, two serine protease inhibitors were tested: PMSF and AEBSF.

PMSF allowed good inhibition of the proteolytic activities of the serine proteases tested. On the other hand, AEBSF only inhibited trypsin. Measurement tests where Listeria (the 3 strains above were tested) is brought into contact with a composition comprising a subtilisin, then AEBSF in a concentration sufficient to cause complete inhibition of subtilisin have shown that different microbiological tests gave lower values, or did not even detect Listeria under the test conditions, unlike the control without the enzymes or when PMSF is added.

Therefore, the inventors chose to incorporate PMSF into the preservative solution to be tested, despite the disadvantages of this molecule.

Unfortunately, even under these conditions, the Vidas® LMX test does identify the presence of Listeria monocytogenes.

### Example 5—Optimisation of the Collection Composition

The inventors then decided to test the effectiveness of a highly diluted collection composition avoiding the compounds identified as harmful as much as possible. In addition, enzymatic activities are reduced, as is the detergent content. Detergents are chosen after laboratory tests based on their compatibility with the life of microorganisms, including Listeria, which has been identified as more sensitive.

First of all, with detergent concentrations of 10% by weight, even in the presence of low enzymatic activity, in the presence of minimal preservative content (e.g. almost completely non-detectable; phenoxyethanol less than 4 ppm) and in the absence of PMSF, Listeria detection tests, such as Vidas®, do not work well (significant underquantification), or do not work in a reproducible manner. Even less specific tests do not work when a Listeria culture is subjected to these different treatments.

Therefore, all the parameters of the composition must be checked, with the problem that an overly high dilution of enzymatic activities risks no longer allowing the removal of microorganisms, while an overly high dilution of detergents risks no longer allowing the even application of the composition or good contact with the biofilm. Therefore, the inventors noticed that, in addition to the quantity of the components, the nature of the components had to be reconsidered.

A diluted formulation was tested comprising about 20% by weight of glycerol, about 0.03% by weight of an anionic detergent of the laureth sulphate type (sodium), 0.4% by weight of sequestrant (carboxymethyl inulin); 0.4% by weight of sodium glycolate; 0.04% by weight of a subtilisin (activity 1 Anson Unit), as well as a lipase; an amylase, a cellulase and a mannanase. The inventors selected enzymes, here commercial enzymes, without preservatives such as potassium sorbate, MIT, OIT, BIT and CIT. When possible, the inventors used commercial enzymes free from phenoxyethanol (cellulase and mannanase), or with a low content of this molecule (subtilisin; 4 g/kg of the enzymatic formulation, lipase; 0.249 g/kg and amylase; 0.233 g/kg). Enzymes other than subtilisin were added in weight contents of between 0.1 and 0.5% (weight of the enzymatic composition: total weight) and the amount of enzyme is typically 1 to 10% by weight relative to the commercial formulation.

Following the use of this composition, the presence of Listeria is detected, and the Vidas® LMX test for Listeria is, ultimately, positive and reproducible. The inventors further noted that, under these diluted conditions, the addition of the protease inhibitor PMSF is not necessary, and even reduces the spread of Listeria.

The inventors consider that the concentration of enzymes other than proteases (here, a subtilisin) can still be somewhat reduced, e.g. the lowest concentration that allows detachment, as well as those of the sequestering agent. In the light of this invention, this can be tested in the laboratory on the 3 strains of Listeria or in other models, including, preferably, more complex ones.

The inventors then tested a possible synergy with the preservative solution (see examples 2 and 3): since the results are already very good, the improvement associated with the addition of the preservative solution is marginal, but it was nevertheless interesting in the case of a test that includes spreading followed by detection by PCR.

1. A method for sampling, removing, collecting and preserving microorganisms on surfaces, for subsequent detection and laboratory analysis comprising the following steps:
performing sterile and calibrated application of a composition for collecting microorganisms comprising one or more surfactants, and at least two enzymes chosen from the group comprising a protease, a polysaccharidase, a laccase, a lipase, a cellulase and a mannanase, and a sequestering agent to a surface to be sampled,
leaving said composition to act on said surface to be sampled for a predetermined period of time to recover a sample of microorganisms,
performing sterile collection of said composition containing said sample of microorganisms,
depositing said composition containing said sample of microorganisms in at least one sterile container,
wherein said surfactants present in said collection composition comprise an anionic surfactant and wherein overall content of said surfactants is between 0.01% and 10% by weight.

2. The method according to claim 1, wherein said calibrated application of said collection composition is a calibrated application of a controlled volume of said collection composition.

3. The method according to claim 2, wherein said calibrated application of a controlled volume of said collection composition is an application of a content of between 0.5 and 5 millilitres of composition per application on a surface of 100 cm².

4. The method according to claim 1, wherein said step of leaving said collection composition to act takes place for a period of at least 10 seconds and at most 15 minutes.

5. The method according to claim 1, wherein said step of sterile collection of said composition containing said sample of microorganisms comprises a first step of bringing at least one means of sterile harvesting of said microorganisms in contact on said surface to be sampled.

6. The method according to claim 1, wherein said step of performing sterile collection of said composition containing said microorganism sample is performed by sterile collection means.

7. The method according to claim 1, further comprising at least one step of storing said sterile container containing said microorganism sample at a temperature below 15° C.

8. The method according to claim 1, wherein the surfactants of said collection composition are present at a content of at least 0.02% by weight.

9. The method according to claim 1, wherein said collection composition comprises at least three enzymes.

10. The method according to claim 1, wherein said collection composition further comprises at least one stabilising agent present at a content of at least 15% relative to the weight of said composition, said stabiliser being chosen from the group of polyols and propylene glycol.

11. The method according to claim 1, wherein said collection composition further comprises at least one sequestering agent at a content of at least 0.1% by weight relative to the weight of said composition, said sequestering agent being a carboxymethyl inulin, a phosphonate, sodium glycolate and mixtures thereof.

12. The method according to claim 1, comprising sterilizing said collection composition previously by a physical treatment chosen from electron beam irradiation, gamma ray irradiation and sterilising filtration.

13. The method according to claim 1, further comprising applying a preservative solution to said sample of microorganisms collected or deposited, for preservation of said sample for subsequent laboratory detection and analysis.

14. The method according to claim 13, wherein said preservative solution comprises at least one polyoxyethylene sorbitan derivative and/or at least one phosphoglyceride derivative.

15. The method according to claim 14, wherein said at least one polyoxyethylene sorbitan derivative is present at a content of at least 4 g/L.

16-17. (canceled)

18. The method according to claim 1, further comprising culturing collected and/or preserved microorganisms.

19. The method according to claim 1, further comprising specifically detecting collected, preserved and/or cultured microorganisms.

20. The method according to claim 19, wherein specific detection is carried out by immunology.

21. The method according to claim 19, wherein specific detection comprises genetic analysis.

22. The method according to claim 19, further comprising performing a biochemical test said biochemical test being an enzymatic test, or involving proteins or enzymes, ATP-metry, NADH measurement, catalase activity measurement and protein residue measurement.

23. The method according to claim 19, wherein the microorganism is Salmonella sp. and/or Listeria sp.
